(19) United States
(12) Patent Application Publication
(10) Pub. No.: US 2025/0195903 A1
(43) Pub. Date: Jun. 19, 2025

(54) DEFIBRILLATOR

(71) Applicant: Nihon Kohden Corporation, Tokyo (JP)

(72) Inventor: Ryosuke Kuno, Saitama (JP)

(21) Appl. No.: 18/974,356

(22) Filed: Dec. 9, 2024

(30) Foreign Application Priority Data
Dec. 18, 2023 (JP) 2023-212986

(51) Int. Cl. A61N 1/39; A61N 1/04

(52) U.S. Cl. CPC A61N 1/3993; A61N 1/0492; A61N 1/3904; A61N 1/3925

(57) ABSTRACT

A defibrillator includes a controller configured to detect a use state of a defibrillator by an operator, and output guidance of a use method of the defibrillator, according to the detected use state. The controller is configured to detect a change in the use state in a period before a timing at which a pair of pads of the defibrillator are attached to a skin of a subject from a timing at which the defibrillator is started up.

### CROSS-REFERENCE TO RELATED APPLICATIONS

This application is based on and claims priority under 35 USC 119 from Japanese Patent Application No. 2023-212986 filed on Dec. 18, 2023, the entire content of which is incorporated herein by reference.

### TECHNICAL FIELD

The presently disclosed subject matter relates to a defibrillator.

### BACKGROUND ART

In the related art, a technique related to a defibrillator such as an automated external defibrillator (AED) is known. For example, JP2016-106914A discloses a defibrillation pad in which electrical connection of an electrical connection pattern having a predetermined electrical resistance value is cut off when a pair of electrode pads are separated and attached to a living body for use. According to such a defibrillation pad, it is possible to detect whether the electrode pad that is accommodated in the package has been used.

In many cases, the operator who uses the defibrillator is unfamiliar with the use of the defibrillator.

### SUMMARY OF INVENTION

Aspect of non-limiting embodiments of the present disclosure relates to provide a defibrillator that enables even an operator who is unfamiliar with the use of the defibrillator to use the defibrillator more reliably and with a more accurate operation procedure.

Aspects of certain non-limiting embodiments of the present disclosure address the features discussed above and/or other features not described above. However, aspects of the non-limiting embodiments are not required to address the above features, and aspects of the non-limiting embodiments of the present disclosure may not address features described above.

According to an aspect of the present disclosure, there is provided a defibrillator including:
a controller configured to:
   detect a use state of a defibrillator by an operator; and
   output guidance of a use method of the defibrillator, according to the detected use state,
in which the controller is configured to detect a change in the use state in a period before a timing at which a pair of pads of the defibrillator are attached to a skin of a subject from a timing at which the defibrillator is started up.

### BRIEF DESCRIPTION OF DRAWINGS

Exemplary embodiment(s) of the present invention will be described in detail based on the following figures, wherein:

FIG. 1 is an external view of an AED according to an embodiment of the presently disclosed subject matter;

FIG. 2 is a block diagram illustrating the configuration of the AED illustrated in FIG. 1;

FIG. 3 is a flowchart illustrating the procedure of guidance when an AED in the related art is used;

FIG. 4 is a graph illustrating the change in impedance between a pair of electrode pads obtained by a detection unit of the AED illustrated in FIG. 2; and

FIG. 5 is a flowchart illustrating an example of the procedure of guidance when the AED according to the embodiment of the presently disclosed subject matter is used.

### DESCRIPTION OF EMBODIMENTS

Hereinafter, a defibrillator according to the presently disclosed subject matter will be described with reference to the drawings. Members having the same reference numerals as those already described in the description of the embodiment will not be described for convenience of description. Further, for convenience of description, dimensions of the members illustrated in the drawings may be different from actual dimensions of the members.

### Configuration of AED

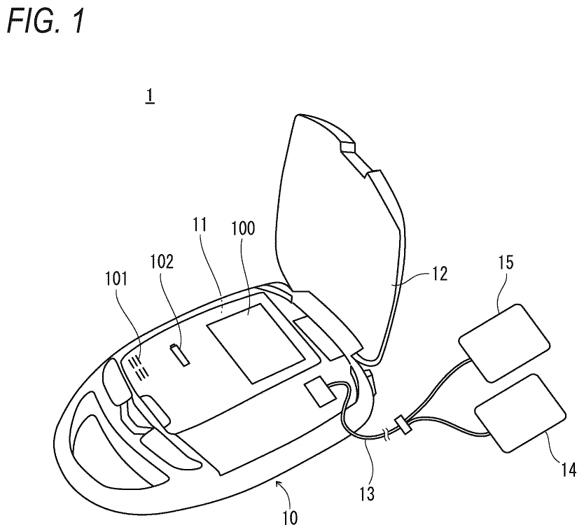

Here, an AED 1 will be described which is an example of a defibrillator according to the presently disclosed subject matter. FIG. 1 is an external view of the AED 1 according to the embodiment of the presently disclosed subject matter. As illustrated in FIG. 1, the AED 1 can include an AED body 10, a battery pack 11, a lid portion 12, a cable 13, and a pair of electrode pads 14 and 15.

The battery pack 11 is configured to supply power for operating the AED body 10 and the electrode pads 14 and 15 to the AED body 10. The battery pack 11 is detachably connected to the back side of the AED body 10.

The lid portion 12 is a structure that covers the AED body 10. In a case where the lid portion 12 changes from the closed state to the open state, the main power supply of the AED 1 is turned on, and the AED 1 is started up. When the lid portion 12 changes from the open state to the closed state, the main power supply of the AED 1 is turned off. The AED 1 is not limited to a configuration in which the main power supply is switched ON/OFF in response to opening and closing of the lid portion 12. For example, a power button configured to switch the main power supply ON/OFF may be provided on the AED body 10.

Each of the electrode pads 14 and 15 can include a gel having viscosity. In a case where the AED 1 is not in use, the electrode pads 14 and 15 are accommodated in a bag, in a state where release paper is attached to each gel. The bag that accommodates the electrode pads 14 and 15 is attached to the inside of the lid portion 12, for example. The operator who uses the AED 1 removes the bag from the lid portion 12 and takes out the electrode pads 14 and 15 from the bag. Then, the operator attaches the gel portion to the skin of a subject who is an injured or sick person in a state in which the electrode pads 14 and 15 are peeled off from the release paper.

The cable 13 electrically connects the AED body 10 and the electrode pads 14 and 15. In a state in which the electrode pads 14 and 15 are attached to the subject, the AED body 10 performs processing such as electrocardiogram analysis and electric shock (hereinafter also referred to as “discharge processing”) on the subject.

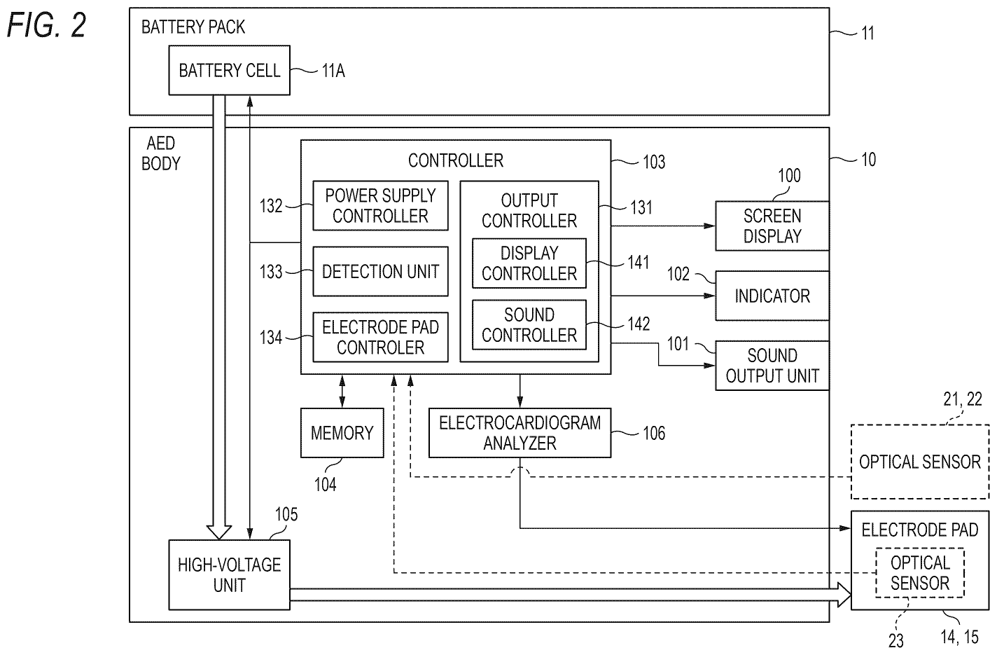

FIG. 2 is a block diagram illustrating the configuration of the AED 1 illustrated in FIG. 1. As illustrated in FIGS. 1 and 2, the AED body 10 can include a screen display 100, a sound output unit 101, and an indicator 102, as a user interface. The battery pack 11 can include a battery cell 11A.

The screen display 100 is a display configured to display an illustration, characters, and the like, and visually displays, to the operator, guidance such as a method for attaching the electrode pads 14 and 15 and a method for processing an electric shock.

The sound output unit 101 is, for example, a speaker, and audibly notifies the operator of voice guidance, a warning sound indicating a state in which a high voltage is charged inside the AED body 10, and the like.

The indicator 102 is implemented by a lamp or the like that can light up or blink, and visually indicates to the operator whether the AED 1 is in a usable state.

As illustrated in FIG. 2, the AED body 10 can further include a controller 103, a memory 104, a high-voltage unit 105, and an electrocardiogram analysis unit 106 in addition to the screen display 100, the sound output unit 101, and the indicator 102. The memory 104 is configured to store a program or the like for controlling various operations of the AED 1.

In a case where the AED 1 is started up, the controller 103 reads and executes the program or the like that is stored in the memory 104, and controls the various operations of the AED 1. More specifically, the controller 103 can include an output controller 131, a power supply controller 132, a detection unit 133, and an electrode pad controller 134.

The output controller 131 can include a display controller 141 and a sound controller 142. The display controller 141 is configured to read the image data relating to the guidance for the operator from the memory 104, and to output the read image data to the screen display 100. Upon receiving the image data that is output from the display controller 141, the screen display 100 displays an image based on the image data.

In order to notify the operator whether the AED 1 is in a usable state, the display controller 141 is configured to output, to the indicator 102, a control signal for a lamp or the like of the indicator 102. The indicator 102 is configured to light up, turns off, and blinks, based on the received control signal.

The sound controller 142 is configured to read sound data such as voice guidance and warning sound for the operator from the memory 104, and to output the read sound data to the sound output unit 101. Upon receiving the sound data output from the sound controller 142, the sound output unit 101 outputs voice or warning sound based on the sound data.

The power supply controller 132 is configured to control the battery cell 11A and the internal capacitor such that the internal capacitor of the high-voltage unit 105 is charged by the battery cell 11A and discharged from the electrode pads 14 and 15.

The detection unit 133 is configured to detect the use state of the AED 1 by the operator. For example, the detection unit 133 is configured to detect the use state of the AED 1 by determining whether the pair of electrode pads 14 and 15 are attached to the skin of the subject. The detection executed by the detection unit 133 will be described later in detail.

In a case where the detection unit 133 detects that the pair of electrode pads 14 and 15 are attached to the subject, the electrode pad controller 134 controls the electrode pads 14 and 15 and the electrocardiogram analysis unit 106 to cause the electrocardiogram analysis unit 106 to execute the electrocardiogram analysis. The electrocardiogram analysis unit 106 executes electrocardiogram analysis of the subject through the electrode pads 14 and 15 by being controlled by the electrode pad controller 134.

### Description of Problem

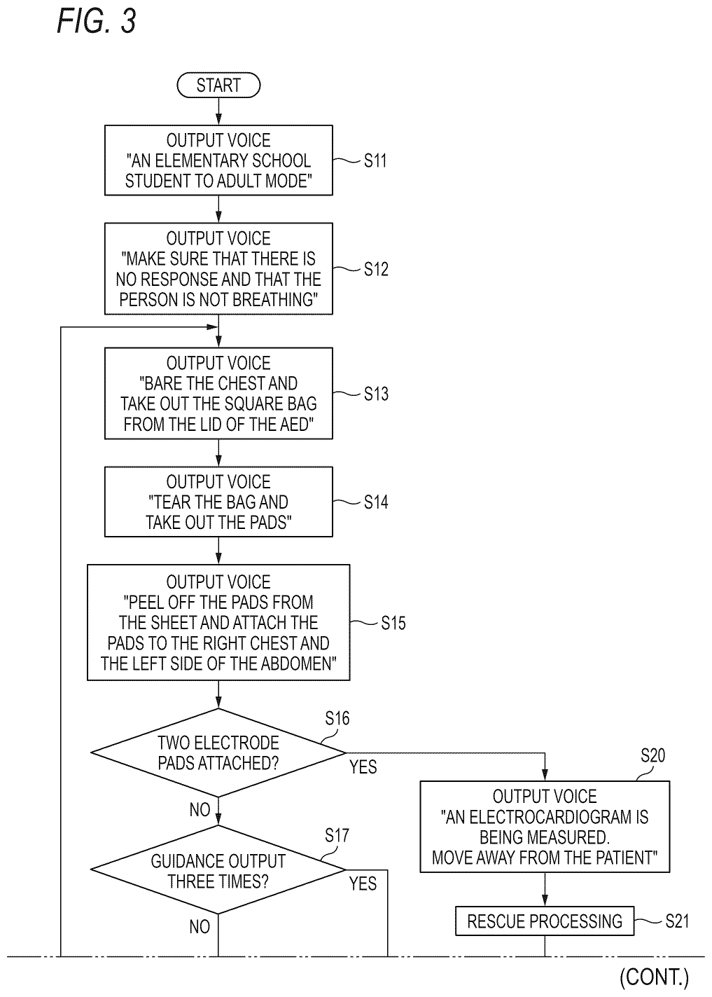

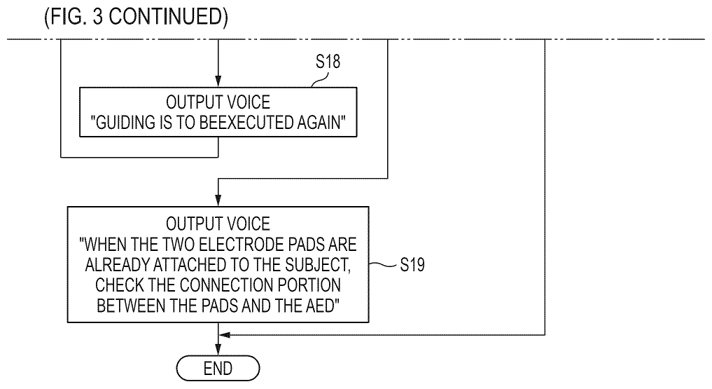

Here, a problem when an AED in the related art is used will be described. FIG. 3 is a flowchart illustrating the procedure of guidance when the AED in the related art is used. As illustrated in FIG. 3, first, when the AED is started up, for example, in order to notify the operator of the current operation mode, the AED outputs a voice “an elementary school student to adult mode” (step S11).

Next, the AED outputs a voice “make sure that there is no response and that the person is not breathing”, for example, as guidance for the operator (step S12), and further outputs a voice “bare the chest and take out the square bag from the lid of the AED” (step S13).

Next, in order to prompt the operator to take out the electrode pads 14 and 15 from the bag, for example, the AED outputs a voice “tear the bag and take out the pads” (step S14).

Next, in order to prompt the operator to peel off the electrode pads 14 and 15 from the release paper and attach the electrode pads 14 and 15 to the subject, for example, the AED repeatedly outputs a voice “peel off the pads from the sheet and attach the pads to the right chest and the left side of the abdomen” five times (step S15). Further, the AED displays a screen on the display according to the content of the voice guidance.

Next, the AED determines whether the two electrode pads are attached to the subject (step S16). Then, when the two electrode pads are not attached to the subject (“NO” in step S16), for example, the AED determines whether the guidance illustrated in step S13 to step S15 has been output three times (step S17).

Next, when the guidance illustrated in step S13 to step S15 has been output three times (“NO” in step S17), for example, the AED outputs a voice “guiding is to be executed again” (step S18), and performs the operations from step S13 onwards again.

On the other hand, assume that the two electrode pads are not attached to the subject (“NO” in step S16) and the guidance illustrated in step S13 to step S15 has been output three times (“YES” in step S17). In this case, for example, in order to prompt the operator to check the connection state of the electrode pads, the AED outputs a voice “when the two electrode pads are already attached to the subject, check the connection portion between the pads and the AED” (step S19), and ends the guidance.

When the AED determines that the two electrode pads are attached to the subject (“YES” in step S16), the AED outputs a voice such as “an electrocardiogram is being measured. Move away from the patient” as guidance for executing an electrocardiogram analysis (step S20). Then, the AED performs the rescue processing for the subject, such as performing the electrocardiogram analysis and the discharge processing for the subject (step S21).

As described above, in the AED in the related art, the guidance from step S13 onwards is repeated until it is detected that the two electrode pads are attached to the subject. Therefore, for example, even in a situation in which the first electrode pad has been attached, that is, in a situation in which the electrode pad has already been taken out from the bag, guidance is output to prompt the operator to remove the bag of the electrode pad from the lid portion or to take out the electrode pad from the bag.

On the other hand, in the AED 1 according to the presently disclosed subject matter, the use state of the AED 1 is detected as follows, and more appropriate guidance is output according to the use state.

### Detection of Change in Use State of AED

Referring again to FIG. 2, the detection unit 133 of the AED 1 according to the embodiment of the presently disclosed subject matter is configured to detect a change in the use state of the AED 1 in a period before the timing at which the pair of electrode pads 14 and 15 are attached to the skin of the subject from the timing at which the AED 1 is started up. For example, the detection unit 133 is configured to constantly obtain the impedance between the pair of electrode pads 14 and 15. Then, the detection unit 133 is configured to detect a change in the use state of the AED 1 in the above-described period, based on the obtained impedance.

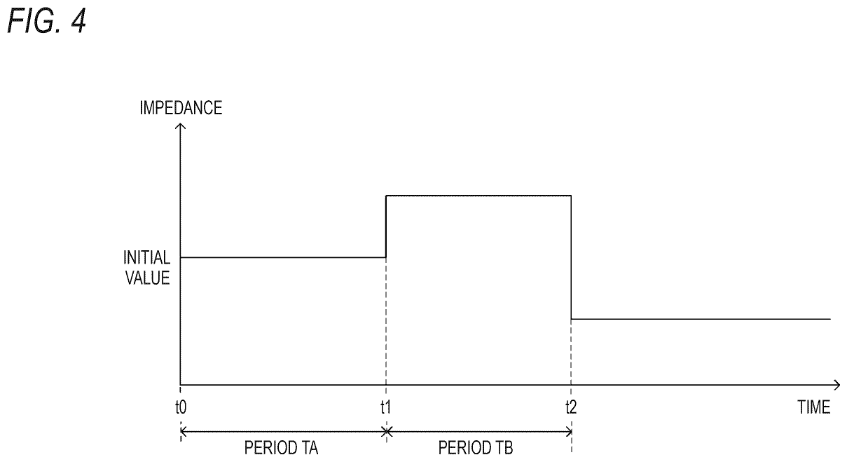

FIG. 4 is a graph illustrating the change in impedance between the pair of electrode pads 14 and 15 illustrated in FIG. 1. In the graph illustrated in FIG. 4, the horizontal axis represents the time, and the vertical axis represents the impedance between the pair of electrode pads 14 and 15.

Referring to FIGS. 2 and 4, it is assumed that the AED 1 is started up at a time t0. At the timing when the AED 1 is started up, since the release paper is attached to the electrode pads 14 and 15, the impedance between the pair of electrode pads 14 and 15 is low. Hereinafter, the value of the impedance at the time t0 will be referred to as an “initial value”.

At a time t1 after the time t0, it is assumed that the impedance rises by a predetermined value or more from the reference value. In this case, the detection unit 133 determines that at least one of the pair of electrode pads 14 and 15 is peeled off from the release paper, as a change in the use state of the AED 1. The reference value is preferably an initial value. Alternatively, the reference value may be, for example, a general absolute value of the impedance when the electrode pads 14 and 15 are not in use. Then, the detection unit 133 is configured to output, to the output controller 131, a detection result indicating that at least one of the electrode pads 14 and 15 is peeled off from the release paper.

In a case where the impedance is equal to or greater than the threshold value, the detection unit 133 may determine that at least one of the pair of electrode pads 14 and 15 is peeled off from the release paper.

At a time t2 after the time t1, it is assumed that the impedance decreases by a predetermined value or more from the reference value. In this case, the detection unit 133 determines that the two electrode pads 14 and 15 are attached to the subject as a change in the use state of the AED 1. As described above, the reference value is preferably an initial value. Alternatively, the reference value may be a value other than the initial value. Then, the detection unit 133 outputs, to the output controller 131, a detection result indicating that the two electrode pads 14 and 15 have been attached to the subject.

The detection unit 133 may determine that the two electrode pads 14 and 15 have been attached to the subject when the impedance is equal to or less than another threshold value lower than the above-described threshold value.

The detection unit 133 may be configured to determine whether to detect that the two electrode pads 14 and 15 is attached to the skin of the subject before a predetermined time elapses from the time t1. In this case, in a case where the detection unit 133 does not detect that both the electrode pads 14 and 15 have been attached to the skin of the subject before the predetermined time elapses from the time t1, the detection unit 133 outputs, to the output controller 131, a detection result indicating that the detection unit 133 does not detect that the electrode pads 14 and 15 have been attached.

### Output of Guidance

The output controller 131 is configured to control the guidance for the operator of the AED 1, according to the use state of the AED 1 that is detected by the detection unit 133. For example, in a case where the detection unit 133 detects that at least one of the electrode pads 14 and 15 is peeled off from the release paper, the output controller 131 executes control such that the content of the guidance is changed before and after the detection.

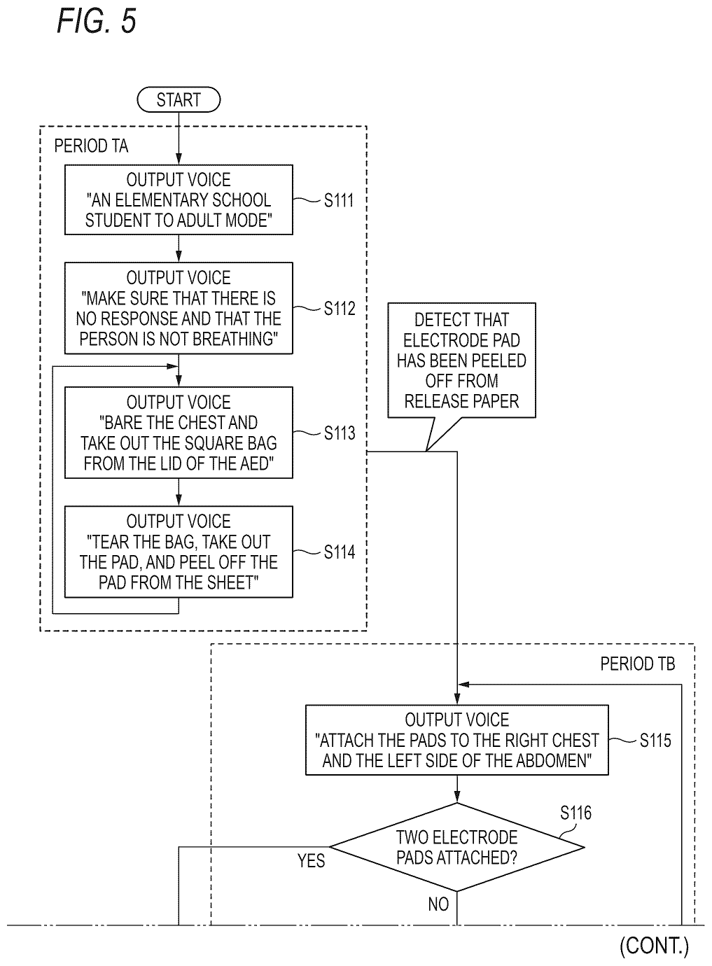

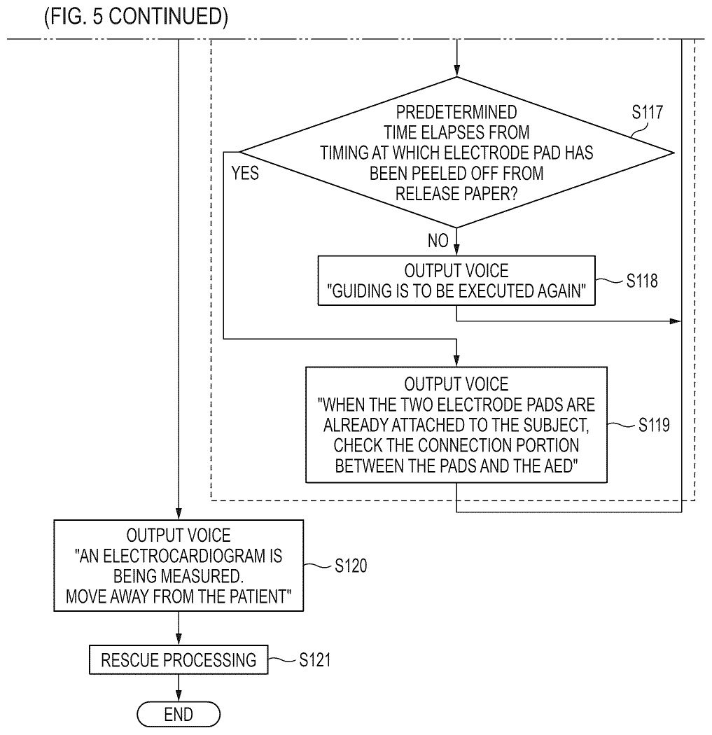

FIG. 5 is a flowchart illustrating an example of the procedure of the guidance when the AED 1 according to the embodiment of the presently disclosed subject matter is used. Here, a period from the time t0 to the time t1 is referred to as a “period TA”. A period from the time t1 to the time t2 is referred to as a “period TB”.

First, in a case where the AED 1 is started up, same or similarly to step S11 illustrated in FIG. 3, for example, in order to notify the operator of the current operation mode of the AED 1, the sound controller 142 outputs a voice “an elementary school student to adult mode” (step S111).

Next, same or similarly to step S12 illustrated in FIG. 3, the sound controller 142 outputs, for example, a voice “make sure that there is no response and that the person is not breathing” (Step S112). Same or similarly to step S13 illustrated in FIG. 3, the sound controller 142 outputs, for example, a voice “bare the chest and take out the square bag from the lid of the AED” (step S113).

Next, in order to prompt the operator to take out the electrode pads 14 and 15 from the bag and to peel off the electrode pads 14 and 15 from the release paper, for example, the sound controller 142 outputs a voice “tear the bag, take out the pad, and peel off the pad from the sheet” (step S114). After the voice output in step S114, a voice “guiding is to be executed again” may be output.

In the period TA from step S111 to step S114, the detection unit 133 constantly detects a change in the use state of the AED 1. Then, in a case where it is not detected that at least one of the electrode pads 14 and 15 is peeled off from the release paper before the predetermined time elapses from the timing at which the voice has been output in step S114, the AED 1 repeats the voice output from step S113 onwards, for example.

On the other hand, it is assumed that at any timing included in the period TA, at least one of the electrode pads 14 and 15 is detected to be peeled off from the release paper. In this case, the period TB begins. At the timing when the period TB begins, the operator has already taken out the bag that accommodates the electrode pads 14 and 15 from the lid portion 12 (see FIG. 1), opened the bag, and taken out the electrode pads 14 and 15 from the bag. Therefore, the sound controller 142 in the AED 1 changes the content of the guidance, and for example, repeatedly outputs a voice “attach the pads to the right chest and the left side of the abdomen” five times (step S115).

Next, the detection unit 133 in the AED 1 checks whether the two electrode pads 14 and 15 are attached to the subject (step S116). Then, when the two electrode pads 14 and 15 are not attached to the subject (“NO” in step S116), the detection unit 133 checks whether a predetermined time has elapsed from the time t1 when it is detected that at least one of the electrode pads 14 and 15 has been peeled off from the release paper (step S117).

Next, when the predetermined time has not elapsed from the time t1 (“NO” in step S117), the sound controller 142 outputs, for example, a voice “guiding is to be executed again” (step S118). Then, the AED 1 performs the operations from step S115 onwards again. The voice output in step S118 may not be performed.

On the other hand, it is assumed that the two electrode pads 14 and 15 are not attached to the subject (“NO” in step S116) and the predetermined time has elapsed from the time t1 (“YES” in step S17). In this case, for example, the sound controller 142 executes control such that guidance prompting the operator to check the connection state of the AED 1 is output from the sound output unit 101, such as “when the two electrode pads are already attached to the subject, check the connection portion between the pads and the AED” (step S119). Then, the AED 1 performs the operations from step S115 onwards again. After the voice output in step S119, the voice “guiding is to be executed again” may be output, and the operations from step S115 onwards may be performed.

On the other hand, when the sound controller 142 determines that the two electrode pads 14 and 15 are attached to the subject (“YES” in step S116), for example, the sound controller 142 outputs a voice “an electrocardiogram is being measured. Move away from the patient” (step S120). Then, the AED 1 performs the rescue processing for the subject, such as performing the electrocardiogram analysis and the electric shock for the subject (step S121).

In addition to the voice output by the sound controller 142 or instead of the voice output by the sound controller 142, the display controller 141 in the AED 1 outputs the image data to the screen display 100 such that a screen corresponding to the guidance content illustrated in step S111 to step S120 is displayed on the screen display 100.

As described above, in the AED 1, a change in the use state of the AED 1 is detected in a period before the timing at which the pair of electrode pads 14 and 15 of the AED 1 are attached to the skin of the subject from the timing at which the AED 1 is started up.

With this configuration, since the use state of the AED 1 by the operator can be detected in detail, more appropriate guidance can be output according to the use state. The more appropriate guidance according to the use state is output, so that the operation to be performed by the operator becomes clear, and the AED 1 can be more reliably used in an accurate operation procedure even if the operator is unfamiliar with the use of the AED 1.

In the AED 1, it is possible to detect that the electrode pad has been peeled off from the release paper to which the electrode pads 14 and 15 are attached, as a change in the use state. In this way, it is possible to detect the operation that is required before the pair of electrode pads 14 and 15 are attached to the subject, so that it is possible to output more appropriate guidance to the operator.

In the AED 1, the content of the guidance is changed before and after it is detected that the electrode pads 14 and 15 have been peeled off from the release paper. With this configuration, for example, unnecessary guidance can be avoided from being repeated for the operation that has already been performed at the timing of peeling off the electrode pads 14 and 15 from the release paper, such as the operation of taking out the bag of the electrode pads 14 and 15 or the operation of tearing the bag and taking out the electrode pads 14 and 15 from the bag.

Here, after at least one of the electrode pads 14 and 15 has been peeled off from the release paper, there is a high chance that the pair of electrode pads 14 and 15 have already been attached to the skin of the subject before a predetermined time elapses. On the other hand, as described above, in the AED 1, even though there is a high chance that the pair of electrode pads 14 and 15 have already been attached, when it is not detected that the pair of electrode pads 14 and 15 have been attached, guidance for prompting the operator to check the connection state of the AED 1 is output. Therefore, it is possible to reduce the operational abnormality of the AED 1 due to the poor connection state.

In the AED 1, a change in the use state of the AED 1 is detected based on the impedance between the pair of electrode pads 14 and 15. With this configuration, since a change in the use state can be detected without attaching a new sensor or the like, an increase in the number of components and an increase in manufacturing costs can be prevented.

In the AED 1, when the impedance becomes equal to or higher than the threshold value or when the impedance rises by a predetermined value or more from the reference value, the detection unit 133 determines that at least one of the electrode pads 14 and 15 has been peeled off from the release paper. With this configuration, it is possible to more accurately detect that at least one of the electrode pads 14 and 15 has been peeled off from the release paper.

In the AED 1, the detection unit 133 determines that the pair of electrode pads 14 and 15 are attached to the subject when the impedance becomes equal to or less than another threshold value or when the impedance decreases by a predetermined value or more from the reference value. With this configuration, it is possible to more accurately detect that the pair of electrode pads 14 and 15 are attached to the subject.

### Modification

The detection unit 133 shown in FIG. 2 may be configured to detect a change in the use state of the AED 1, using other types of measurement results, instead of the measurement result of the impedance between the electrode pads 14 and 15 or in addition to the measurement result of the impedance.

For example, in a state where the lid portion 12 illustrated in FIG. 1 is opened, the bag that accommodates the electrode pads 14 and 15 is attached to the lid portion 12 as described above. It is assumed that an optical sensor 21 is attached to the surface of the bag in contact with the lid portion 12. In this case, the detection unit 133 is configured to detect that the bag has been taken out from the lid portion 12 of the AED 1 based on the measurement value obtained by the optical sensor 21.

For example, the optical sensor 22 may be attached to the release paper of the electrode pads 14 and 15. In this case, the detection unit 133 is configured to detect that the electrode pads 14 and 15 have been peeled off from the release paper and that the electrode pads 14 and 15 to which the release paper is attached have been taken out from the bag based on the measurement value obtained by the optical sensor 22.

For example, the optical sensor 23 may be attached to the surface of the electrode pads 14 and 15 to which the release paper is attached. In this case, the detection unit 133 is configured to detect that the electrode pads 14 and 15 have been peeled off from the release paper or that the electrode pads 14 and 15 are attached to the subject, based on the measurement value obtained by the optical sensor 23.

Specifically, in a case where the intensity of the brightness that is measured by the optical sensor 23 exceeds a first threshold value, the detection unit 133 may determine that the electrode pad 14 or the electrode pad 15 to which the optical sensor 23 is attached has been taken out from the bag. In a case where the intensity of the brightness that is measured by the optical sensor 23 exceeds the first threshold value and then falls below a second threshold value that is smaller than the first threshold value, the detection unit 133 may determine that the electrode pad 14 or the electrode pad 15 to which the optical sensor 23 is attached is attached to the subject. Since the optical sensor 23 can be provided on each of the electrode pad 14 and the electrode pad 15, it is possible to detect attachment to the subject in units of one pad in addition to attachment and detachment of the release paper in units of one pad.

As described above, in the AED 1 according to the modification, the change in the use state of the AED 1 can be detected based on the measurement values obtained by the optical sensors 21, 22, and 23, such as the optical sensor 23 attached to the electrode pads 14 and 15. With this configuration, for example, when the electrode pads 14 and 15 are attached to the skin of the subject, since a change occurs in the measurement value that is obtained by the optical sensor 23 attached to the electrode pads 14 and 15, it is possible to easily detect that the electrode pads 14 and 15 have been attached to the skin of the subject.

In the AED 1 according to the modification, the use state of the AED 1 by the operator can be detected in even more detail. Therefore, for example, until the bag of the electrode pads 14 and 15 has been taken out from the lid portion 12, it is possible to output more appropriate guidance according to the use state of the operator, such as repeating a voice “take out the bag from the lid of the AED”.

The foregoing description of the exemplary embodiments of the present invention has been provided for the purposes of illustration and description. It is not intended to be exhaustive or to limit the invention to the precise forms disclosed. Obviously, many modifications and variations will be apparent to practitioners skilled in the art. The embodiments were chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

What is claimed is:

1. A defibrillator comprising:
a controller configured to:
detect a use state of a defibrillator by an operator; and
output guidance of a use method of the defibrillator, according to the detected use state,
wherein the controller is configured to detect a change in the use state in a period before a timing at which a pair of pads of the defibrillator are attached to a skin of a subject from a timing at which the defibrillator is started up.

2. The defibrillator according to claim 1,
wherein the controller is configured to detect, as the change in the use state, that at least one of the pair of pads is peeled off from a release paper to which each of the pair of pads is attached.

3. The defibrillator according to claim 2,
wherein the controller is configured to change a content of the guidance before and after the controller detects that at least one of the pair of pads is peeled off from the release paper.

4. The defibrillator according to claim 1,
wherein the controller is further configured to detect that the pair of pads are attached to the skin of the subject, and
in a case where the controller does not detect that the pair of pads are attached to the skin of the subject before a predetermined time elapses after the controller detects that at least one of the pair of pads is peeled off from the release paper, the controller is configured to output the guidance to prompt an operator to check a connection state of the defibrillator and each of the pair of pads.

5. The defibrillator according to claim 1,
wherein the controller is configured to detect the change in the use state, based on an impedance between the pair of pads.

6. The defibrillator according to claim 5,
wherein, in a case where the impedance becomes equal to or greater than a threshold value or in a case where the impedance rises by a predetermined value or more from a reference value, the controller determines that at least one of the pair of pads is peeled off from a release paper.

7. The defibrillator according to claim 5,
wherein, in a case where the impedance becomes equal to or less than a threshold value or in a case where the impedance decreases by a predetermined value or more from a reference value, the controller determines that the pair of pads is attached to the subject.

8. The defibrillator according to claim 1,
wherein the controller is configured to detect the change in the use state, based on a measurement value obtained by an optical sensor attached to each of the pair of pads.

9. The defibrillator according to claim 8,
wherein the controller is configured to detect, as the change in the use state, that at least one of the pair of pads is peeled off from a release paper or that the pads are taken out from a bag, based on the measurement value obtained by the optical sensor.
